(19) United States
(12) Patent Application Publication
(10) Pub. No.: US 2009/0068101 A9
(43) Pub. Date: Mar. 12, 2009

(54) A1 Adenosine Receptor Antagonists

(75) Inventors: Constance Neely Wilson, Raleigh, NC (US); John J. Partridge, Chapel Hill, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: ENDACEA, INC., RESEARCH TRIANGLE PARK, NC (US)

(21) Appl. No.: 10/560,853

(22) PCT Filed: Jun. 7, 2004
(86) PCT No.: PCT/US04/18171

§ 371 Date: Mar. 23, 2007

Related U.S. Application Data
(60) Provisional application No. 60/476,967, filed on Jun. 9, 2003.

(51) Int. Cl. A61K 31/522

(52) U.S. Cl. 424/1.65; 514/263.3; 544/265

(57) ABSTRACT

This invention relates to compounds of formula (I): wherein R₁ is a branched or straight chain C₁-C₆ alkyl; R₂ is of the formula (II), wherein n is an integer ranging from 1 to 8; R₅ is H or (CH₂)ₚCH₃, and R₆ is H or (CH₂)ₘOH, wherein p is an integer ranging from 1 to 7 and m is an integer ranging from 1 to 8; R₃ is of the formula (III), wherein q is an integer ranging from 1 to 8; and R₇ is selected from the group consisting of H, OH, NH₂, (CH₂)ₜOH, and R₉COOH; wherein R₉ is a straight or branched chain alkylene or alkenylene group having 1 to 8 carbon atoms, and t is an integer ranging from 1 to 8; R₄ is of the formula (IV), wherein r is an integer ranging from 1 to 8 and R₈ is selected from the group consisting of H, OH, (CH₂)_{f}NH₂, (CH₂)ₛOH, and R₁₀COOH; wherein f is 0 or f and s are independently integers ranging from 1 to 8; and, R₁₀ is a C₁-C₈ straight or branched chain alkylene or alkenylene; and; salts, solvates, and hydrates thereof. The present invention further provides methods of preparing the compounds of formula (I) and their use as therapeutic agents and diagnostic agents.

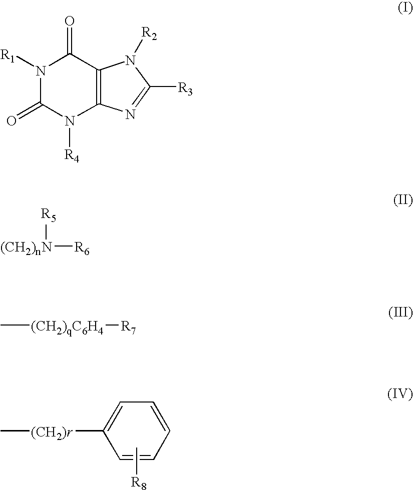

### RELATED APPLICATIONS

This application claims the benefit of U.S. provisional patent application Ser. No. 60/476,967 filed Jun. 9, 2003, the disclosure of which is incorporated by reference herein in its entirety.

### FIELD OF THE INVENTION

The present invention relates to novel compounds useful as A₁ adenosine receptor antagonists.

### BACKGROUND OF THE INVENTION

Adenosine receptors are involved in a vast number of peripheral and central regulatory mechanisms such as, for example, vasodilation, cardiac depression, inhibition of lipolysis, inhibition of insulin release and potentiation of glucagon release in the pancreas, and inhibition of neurotransmitter release from nerve endings.

In general, adenosine receptors can be divided into two main classes, A₁ receptors which can inhibit, and A₂ receptors which can stimulate adenylate cyclase activity. One of the best known classes of adenosine receptor antagonists are the xanthines which include caffeine and theophylline. See e.g., Müller et al., J. Med. Chem. 33: 2822-2828 (1990).

In general, many of these antagonists often exhibit poor water solubility, and low potency or lack of selectivity for adenosine receptors. Additionally, selective analogues of adenosine receptor antagonists have been developed through the “functionalized congener” approach. Analogues of adenosine receptor ligands bearing functionalized chains have been synthesized and attached covalently to various organic moieties such as amines and peptides. Attachment of the polar groups to xanthine congeners has been found to increase water solubility. Nonetheless, such developments have yet to fully address problems associated with potency and selectivity.

### SUMMARY OF THE INVENTION

In one aspect, the invention is a compound of the general formula (I): 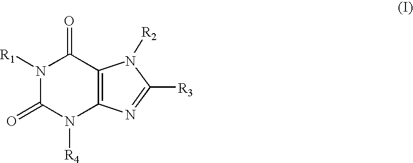
wherein
R₁ is a branched or straight chain C₁-C₈ alkyl,
R₂ is of the formula (II), 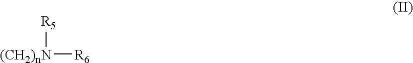
   wherein n is an integer ranging from 1 to 8; R₅ is H or (CH₂)ₚCH₃, and R₆ is H or (CH₂)ₘOH,
      wherein p is an integer ranging from 1 to 7 and m is an integer ranging from 1 to 8;
R₃ is of the formula (III),
   —(CH₂)_{q}C₆H₄—R₇ (III)
   wherein q is an integer ranging from 1 to 8; and R₇ is selected from the group consisting of H, OH, NH₂, (CH₂)ₜOH, R₉COOH;
      wherein R₉ is a straight or branched chain alkylene or alkenylene group having 1 to 8 carbon atoms, and t is an integer ranging from 1 to 8;
R₄ is of the formula (IV), 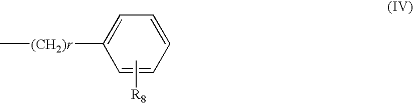
   wherein r is an integer ranging from 1 to 8 and R₈ is selected from the group consisting of H, OH, (CH₂)_{f}NH₂, (CH₂)ₛOH, and R₁₀COOH
      wherein f is 0 or f and s are independently integers ranging from 1 to 8; and,
      R₁₀ is a C₁-C₈ straight or branched chain alkylene or alkenylene; and; salts, solvates, and hydrates thereof.

A second aspect is a method of treating A₁ adenosine receptor related disorders in a mammal, including a human, comprising administering an effective amount of a compound of formula (I) or a salt, solvate or prodrug to the mammal in need thereof.

A third aspect provides a pharmaceutical composition which comprises a compound of formula (I) and a pharmaceutically acceptable carrier.

A fourth aspect provides for diagnostic assay-type probes of a compound of formula (I), wherein the probes are labeled or conjugated with radioactive or non-radioactive material.

A fifth aspect is the use of a compound of formula (I) as an imaging agent in diagnostic procedures such as MRI and PET.

A sixth aspect is the use of a compound of formula (I) in a cell or receptor based assay.

A seventh aspect is the preparation of a compound of formula (I).

An eighth aspect is the preparation of a compound of formula (I) for use as a medicament.

### DETAILED DESCRIPTION OF EMBODIMENTS OF THE INVENTION

The invention will now be described more fully hereinafter, in which embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all technical and scientific terms used herein have the same meaning as commonly understood by one of ordinary skill in the art to which this invention belongs.

All publications, U.S. patent applications, U.S. patents and other references cited herein are incorporated by reference in their entireties.

In one embodiment of a compound according to formula (I), R₁ is C₃ alkyl; R₅ is CH₃(CH₂)ₚ wherein p is 1; R₆ is (CH₂)ₘOH wherein m is 2; R₇ is H; R₈ is NH₂; f is 0; n is 2; q is 1; and r is 2.

In another embodiment of a compound according to formula (I) , R₁ is C₃ alkyl; R₅ is CH₃(CH₂)ₚ wherein p is 1; R₆ is H; R₇ is NH₂; R₈ is NH₂; f is 0; n is 2; q is 1; and r is 2.

In another embodiment of a compound according to formula (I), R₁ is C₃ alkyl; R₅ is CH₃(CH₂)ₚ wherein p is 1; R₆ is H; R₇ is H; R₈ is NH₂; f is 0; n is 2; q is 1; and r is 2.

In another embodiment of a compound according to formula (I), R₁ is C₃ alkyl; R₅ is CH₃(CH₂)ₚ wherein p is 1; R₆ is H; R₇ is H; R₈ is (CH₂)ₛOH, wherein s is 2 and R₁₀COOH, wherein R₁₀ is CH═CH; n is 2; q is 1; and r is 2.

In another embodiment of a compound according to formula (I), R₁ is C₃ alkyl; R₅ is CH₃(CH₂)ₚ wherein p is 1; R₆ is H; R₇ is R₉COOH, wherein R₉ is CH═CH and (CH₂)ₜOH, wherein t is 2; R₈ is NH₂; f is 0; n is 2; q is 1; and r is 2.

The compound of formula (I) may form salts with both organic and inorganic acid and bases. Likewise, the compounds of formula (I) may form solvates including hydrates. All salts and solvates of the compounds of formula (I) are within the scope of the present invention. While pharmaceutically acceptable salts and solvates are useful for the treatment of mammal including humans, non-pharmaceutically salts and solvates may be useful as chemical intermediates, and thus, are within the scope of the present invention. Examples of suitable acids for pharmaceutically acceptable salt formation include, but are not limited to, hydrochloric, sulfuric, phosphoric, acetic, citric, oxalic, malonic, salicylic, ascorbic, maleic, methanesulfonic, benzenesulfonic, p-toluenesulfonic and the like. Any of the amine acid addition salts may also be used. The salts are prepared by contacting the free base form of the compound with an appropriate amount of the desired acid in a manner known to one skilled in the art.

Examples of suitable bases for pharmaceutically acceptable salt formation include, but are not limited to, ammonium hydroxide, sodium hydroxide, sodium carbonate, sodium bicarbonate, potassium hydroxide, calcium hydroxide, ammonia, organic amines such as triethylamine, and the like. The salts may be prepared by contacting the free acid form of the compound with an appropriate amount of the desired base in a manner known to one skilled in the art. An example-of-a suitable solvate is a hydrate. Solvates may be prepared by any appropriate method of the art.

The compounds of formula (I) may be administered per se or in the form of acid or basic salts, hydrates, solvates and pro-drugs thereof, in accordance with known techniques, to carry out the methods- described herein.

Active compounds of the invention may be provided in the form of prodrugs. The term “prodrug” refers to compounds that are transformed in vivo to yield the parent compound of the above formulae, for example, by hydrolysis in blood. A thorough discussion is provided in T. Higuchi and V. Stella, Prodrugs as Novel Delivery Systems, Vol. 14 of the A.C.S. Symposium Series and in Edward B. Roche, ed., Bioreversible Carriers in Drug Design, American Pharmaceutical Association and Pergamon Press, 1987. See also U.S. Pat. No. 6,680,299. Examples include, but are not limited to, a prodrug that is metabolized in vivo by a subject to an active drug having at least some of the activity of the active compounds as described herein, wherein the prodrug is an ester of an alcohol or carboxylic acid group, if such a group is present in the compound; an acetal or ketal of an alcohol group, if such a group is present in the compound; an N-Mannich base or an imine of an amine group, if such a group is present in the compound; or a Schiff base, oxime, acetal, enol ester, oxazolidine, or thiazolidine of a carbonyl group, if such a group is present in the compound, such as described in U.S. Pat. No. 6,680,324 and U.S. Pat. No. 6,680,322.

The compounds of the present invention can be useful in diagnostic assays. Accordingly, the invention also provides A₁ adenosine receptor antagonist compounds with radioactive or non-radioactive labels suitable for executing such assays. Labeled compounds are useful as assay-type probes or conjugates, and to obtain quantitative binding measurements of the A₁ adenosine 30 receptor antagonist compounds. As used herein, the term “assay-type probes” refers to those materials which are useful for enhancing the selectivity of the quantitative analysis of the A₁ adenosine receptor compounds of the invention.

Examples of such assay-type probes and their diagnostic uses are described in Jacobson, et al., U.S. Pat. No. 5,248,770 ('770). The probes are useful because they have little adverse effect on the affinity of the compounds of the present invention. Nuclear markers (also referred to a “labels”) include, but are not limited to, nuclear spin markers, e.g. a ¹⁹F MRI probe, radioactive markers, e.g., ¹⁸F, ¹¹C, ¹⁵N, ¹²⁵I, and ³H (tritium) isotope marker, and complexes of metal atoms or metal ions and chelating agents. Typically the metal or metal ion in the complex will have a heavy, radioactive nucleus. The marker atoms may be chemically bonded to, or complexed, e.g. chelated, with, a compound of formula (I) or may be one of the integral carbon or heteroatom of a compound of formula (I).

Such labeled compounds can be used for in vitro or in vivo imaging of adenosine receptors, especially in tissues, including but not limited to the brain, heart, liver, kidney, and lungs to obtain quantitative measurements of adenosine receptors and determine the distribution and regional binding characteristics of adenosine receptors in tissue. These assay-type probes may be used, inter alia, in connection with such diagnostic techniques as magnetic resonance imaging (MRI) and positron emission tomography (PET). See, for example, Myer, et al., Quantification of cerebral A1 Adenosine Receptors in Humans Using [18F]CPFPX and PET. J Cerebral Blood Flow & Metabolism 24:323-333, 2004 and Wakabayashi, et al., A PET Study of Adenosine A1 Receptor in the Anesthetized Monkey Brain, Nuclear Med & Biol 27:401-406, 2000. An exemplary metal ion is a radioactive isotope of technetium or indium. An exemplary chelating agent is diethylenetriamine pentaacetic acid.

Various non-radioactive materials can be used in labeling the present A₁ adenosine receptor compounds. Numerous examples are presented in U.S. Pat. No. 5,248,770. Biotin is a well known non-radioactive label for such probes, as described in R. W. Old et al. Principals of Gene Manipulation, 4th ed: 328-331 (1989). To facilitate labeling the compounds with biotin or any other appropriate label, a spacer component or moiety may be added to a compound of the present invention by any suitable method taught in the art, e.g. see U.S. Pat. No. 5,248,770. Exemplary spacer moieties include, but are not limited to, an oligopeptide, triglycidyl, N-hydroxysuccinimide ester, succinimidyl-thiohexane (6-thiohexyl-3-amidocarboxypropanoyl), succinimidyl hexamethyleneamine (6-aminohexyl-3-amidocarboxypropanoyl), succinimidyl-cadaverine (5-aminopentyl-3-amidocarboxypropanoyl), and succinimidyl-hexylmaleimide (6-N-maleimidohexyl-3-amidocarboxypropanoyl).

A non-radioactive label, e.g., biotin, may be bonded to any suitable linkage provided by substituents on the compound structure in accordance with any suitable technique taught in the art. For example, referring to compound (I) as defined herein, biotin may be bonded to the hydroxy group on R₆ when the compound contains (CH₂)ₘOH at R₆ with m defined herein; to the amino group present on either of R₇ or R₈ when NH₂ is contained at these positions; or to the carboxyl group present when R₇ and R₈ are R₉COOH or R₁₀COOH respectively, with R₉ and R₁₀ defined herein. Additionally, the biotin may be bonded to a hydroxyl group present on R₈, when R₈ is (CH₂)ₛOH with s being defined herein. Biotin may also be bonded to R₇, when R₇ is (CH₂)ₜOH with t being defined herein. The biotin-labeled probes may be detected through appropriate and known analytical techniques

Fluorescent compounds, typically fluorescent dyes, may also be employed as a non-radioactive labels and are applied to appropriate locations on the compounds of the invention as described above. Such dyes include, but are not limited to, tetramethylrhodamine, fluorescein isothiocyanate, Cy3, (see Waggoner, et al., U.S. Pat. No. 5,268,486, Dec. 7, 1993) or Cy3B (see Waggoner et al., U.S. Pat. No. 6,133,445, Oct. 17, 2000) and mixtures thereof. Other non-radioactive materials include for example, nitrobenzoxadiazole; 2,2,6,6-tetramethyl-piperindinyloxy-4-isothiocyanate; luminescent dyes; obelin; and mixtures thereof, which may be applied in an analogous manner as fluorescent compounds.

The skilled artisan will appreciate that also within the scope of the invention is the use of a compound of formula (I) marked with a radioactive or non-radioactive label in in vitro assays. For example, such marked compounds may be used in clinical cell-based assays and in receptor-based assays. Such assays include, but are not limited to, radioligand binding assays, high throughput screening assays, and flow cytometry based assays, for example fluorescence-activated cell sorting (FACS) based assays. Examples of such assays include, but are not limited to, radioimmunoassay and enzyme-linked immunosorbent assays (ELISA) (see, e.g., Nelson, et al., Lehninger Principles of Biochemistry, 231, Worth, N.Y., (2000).

The invention is also directed to pharmaceutical compositions which include compounds of the present invention and a pharmaceutically acceptable carrier. The pharmaceutical compositions described herein can be prepared by any applicable method of the art. The pharmaceutical composition is particularly useful in applications relating to organ preservation in vivo or in situ, perfusion of an isolated organ either removed or contained within the body (e.g., when an organ is transported for transplantation), cardiopulmonary bypass, perfusion of an extremity or limb, and the like. The compounds may be used in intra-articular, intra-thecal, gastrointestinal, and genital urinary applications, as well as in any cavity or lumen such as, for example, the thoracic cavity or ear canal.

While the present invention is intended primarily for the treatment of human subjects, it will be appreciated that other subjects, particularly mammalian subjects such as dogs, cats, horses, rabbits, etc., can also be treated by the methods of the present invention for veterinary purposes

The pharmaceutical compositions may be employed, as an example, in oral dosage form as a liquid composition. Such liquid compositions can include suspension compositions or syrup compositions and can be prepared with such carriers as water; a saccharide such as sucrose, sorbitol, fructose, and the like; a glycol such as polyethyleneglycol, polypropyleneglycol, and the like; an oil such as sesame oil, olive oil, soybean oil, and the like; an antiseptic such as p-hydroxy-benzoic acid esters and the like; and a flavor component such as a fruit flavor or a mint flavor.

The pharmaceutical compositions may also be in the form of powder, tablets, capsules, and tablets and can be prepared with various carriers. Suitable carriers include, but are not limited to, lactose, glucose, sucrose, mannitol, and the like; disintegrators such as starch, sodium alginate, and the like; binders such as polyvinyl alcohol, hydroxypropyl cellulose, gelatin; and the like; surfactants such as, for example, fatty acid esters; and plasticizers such as, for example, glycerins. The composition of the present invention is especially useful when applied sublingually. It should be noted that in the preparation of the tablets and capsules, a solid pharmaceutical carrier is used. Advantageously, the pharmaceutical compositions may be used in the form of, for example, eye drops or an aerosol.

Other types of pharmaceutical compositions may be employed in the form of a suppository, a nasal spray, and an injectable solution. These compositions are prepared using appropriate aqueous solutions which may include, but are not limited to, distilled water, and saline and buffer additives. Other components may be employed such as organic materials including neutral fatty bases. Additionally, the pharmaceutical compositions may be utilized in a transdermal application.

Biopolymers may be used as carriers in the above pharmaceutical compositions. Exemplary biopolymers may include, for example, proteins, sugars, lipids, or glycolipids. See, e.g., PCT Application WO 02/095391 (Published Nov. 22, 2002).

The A₁ receptor antagonists of the present invention are particularly useful as, for example, anti-allergenics, anti-inflammatory agents, CNS stimulants, diuretics, anti-asthmatics, cardiotonics, coronary yasodilators, and anti-tussives and as agents for the treatment of viral or retroviral infections and immune deficiency disorders such as acquired immunodeficiency syndrome (AIDS).

The present invention also provides a method of treating A₁ adenosine receptor related disorders, such disorders including, but not limited to, congestive heart failure, hypertension, such as systemic hypertension and pulmonary hypertension, ischemia-reperfusion organ injury, endotoxin-related tissue injury, renal failure, Alzheimer's disease, depression, obesity, asthma, diabetes, cystic fibrosis, allergic conditions, including, but not limited to allergic rhinitis and anaphylactic shock, autoimmune disorders, inflammatory disorders, chronic obstructive pulmonary disorders, chronic cough, coronary artery disease, biliary colic, postoperative ileus, fibrosis, sclerosis, Adult Respiratory Distress Syndrome (ARDS), Acute Lung Injury (ALI), Severe Acute Respiratory Syndrome (SARS), septicemia, substance abuse, drug dependence, Parkinson's disease, and acquired immunodeficiency syndrome (AIDS).

The dosage of the active agent will depend upon the condition being treated, the age and condition of the subject, the route of administration, etc. In general, the dosage can be determined in accordance with known techniques. In one embodiment, the dosage of the active agent may, for example, be from 1 or 10 to 300 or 800 mg per adult subject.

The compounds described herein may be used alone or in combination with other compounds for the treatment of the disorders described herein, including, but not limited to, those compounds described in PCT Application, WO 03/103675, published Dec. 18, 2003.

Thus, according to other embodiments of the invention, the present invention relates to a method of treating A₁ adenosine receptor-related disorders, comprising concurrently administering:

(a) an A₁ adenosine receptor antagonist as described above, or a pharmaceutically acceptable salt thereof; with

(b) an additional active agent such as a compound selected from the group consisting of fluticasone propionate, salmeterol, theophylline, A₁ adenosine receptor antagonists, A₂ₐ adenosine receptor agonists, A_{2b} adenosine receptor antagonists, A₃ adenosine receptor antagonists, P_{2y} purinoceptor agonists, and P₂ₓ purinoceptor antagonists, and combinations thereof, in an effective amount to treat the A₁ adenosine receptor-related disorder.

As used herein, “effective amount” or “effective therapeutic amount” refers to a nontoxic but sufficient amount of the compound to provide the desired pharmacological effect, including but not limited to, improvement in the condition of the subject (e.g., in one or more symptoms), delay in the progression of the condition, prevention or delay of the onset of the disease or illness, etc.

As pointed herein, the exact amount required will vary from subject to subject, depending on age, general condition of the subject, the severity of the condition being treated, the particular biologically active agent administered, and the like. An appropriate “effective” amount in any individual case may be determined by one of ordinary skill in the art by reference to the pertinent texts and literature and/or by using routine experimentation.

An effective amount of a prodrug of the present invention is the amount of prodrug that must be metabolized within the body or a mammal, such as a human, to yield and an effective amount of a compound of formula (I).

The present invention relates to a method of treating A₁ adenosine receptor-related disorders, comprising concurrently administering an A₁ adenosine receptor antagonist as described above with at least one additional active agent such as described above effective to treat A₁ adenosine receptor-related disorders, wherein the A₁ adenosine receptor-related disorder is as described above.

Administration of compounds in combination may be carried out in like manner as described above, with the active compound and the additional active agent being administered in the same or different carrier. Pharmaceutical formulations containing such combinations of active agents may also be prepared in like manner as described above.

The compounds of formula (I) may be prepared by any applicable method of the art. Conveniently, they may be prepared by the method of scheme 1.

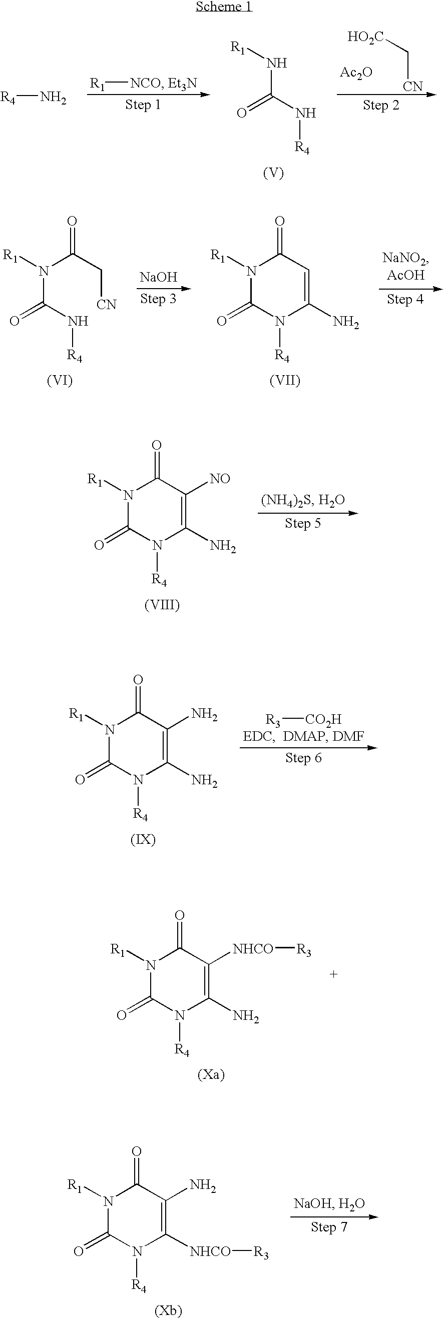

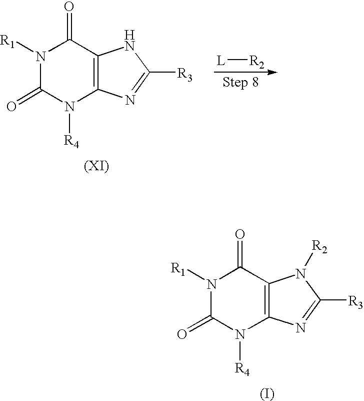

In Step 1, R₄NH₂ is condensed with R₁NCO in the presence of a base such as triethylamine to yield the substituted ureas of formula (V), which in turn, in Step 2, are reacted with cyanoacetic acid to yield the compounds of formula (VI). In Step 3, in the presence of a strong base such as NaOH, the compounds of formula (VI) are converted into the compounds of formula (VII). In Step 4, treatment of the compounds of formula (VII) with NaNO₂ under acidic conditions, such as in the presence of acetic acid, gives the compounds of formula (VIII).

In Step 5, the compounds of formula (IX) are prepared by reducing the compounds of formula (VIII) with (NH₂)₂S. The conversion of the compounds of formula (IX) to the compounds of formula (Xa) and (Xb) is realized in Step 6 by treatment with R₃COOH in the presence of amide coupling agents such as 1-(3-dimethylaminopropyl)-3-ethylcarbodiimide hydrochloride (EDC) and 4-(dimethylamino)pyridine (DMAP) in dimethylformamide (DMF) solvent. In Step 7, the compounds of formulas (Xa) and (Xb) are cyclized in the presence of a strong base such as NaOH to yield the compounds of formula (XI) Finally, in Step 8, the compounds of formula (XI) are reacted-with a compound of the formula L-R2, wherein L is a leaving group (such as a halide), to give a compound of formula (I).

Those skilled in the art of organic chemistry will recognized that at various stages in the preparation of the compounds of the formulae described herein, intermediates may have to be protected and subsequently deprotected or converted to their final form. For example, if R3 contains a nitro group, the final product must be subjected to reducing conditions by methods well known in the art to convert it to an amino group. Additionally, it should be noted that steps comprising the methods provided herein can be performed independently or at least two steps can be combined without departing from the teachings of the present invention.

The present invention is explained in greater detail in the following non-limiting Examples.

### EXAMPLE 1

### Synthesis of 5,6-Diamino-1-[2-(4-nitrophenyl)ethyl]-3-propyluracil (6)

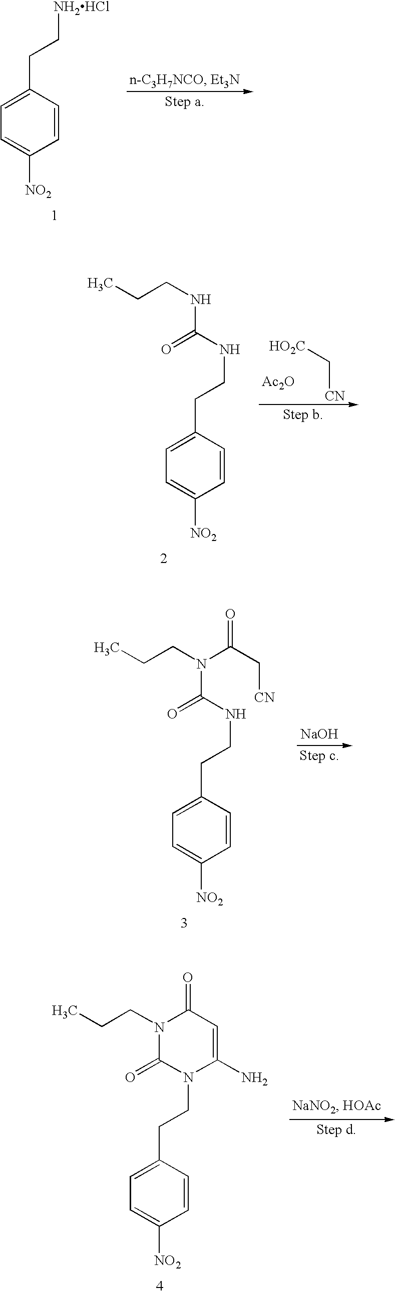

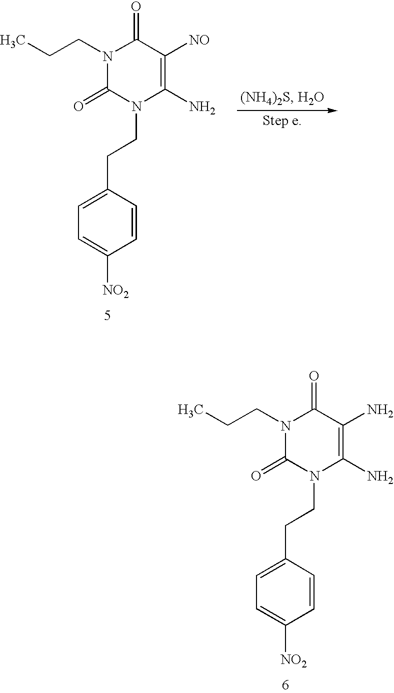

### Step a: Conversion of 4-Nitrophenethylamine Hydrochloride (1) to 1-[2-(4-Nitrophenyl)ethyl]-1′-propylurea (2)

To a slurry of 777 gm of 4-nitrophenethylamine hydrochloride (1) and 11.2 L of toluene was added slowly, 620 mL of triethylamine and this mixture was stirred 10 for 30 min. at room temperature. To this suspension was then added slowly, 398 mL of n-propyl isocyanate, and the mixture was stirred overnight at room temperature to give a solid precipitate. The heterogeneous mixture was filtered and the isolated solids were washed with 1.5 L of toluene and then air dried. The 2.3 kg of crude product was stirred with 6 L of water to dissolve residual triethylamine hydrochloride. The solids were isolated by filtration and air dried. This material was dissolved in 4 L of absolute ethanol and 1 L of water was added to induce crystallization. The solids were filtered, washed with 2 L of 1:1 ethanol-water and air dried to yield a first crop of 880 gm of 1-[2-(4-nitrophenyl)ethyl]-1′-propylurea (2). The recrystallization mother liquors yielded an additional 39.8 gm of 1-[2-(4-nitrophenyl)ethyl]-1′-propylurea (2).

### Step b: Conversion of 1-[2-(4-Nitrophenyl)ethyl]-1′-propylurea (2) to 1′-Cyanoacetyl-1-[2-(4-nitrophenyl)ethyl-1′-propylurea (3)

A thick mixture of 920 gm of 1-[2-(4-nitrophenyl)ethyl]-1′-propylurea (2) and 1.0 L of acetic anhydride was stirred and warmed to ca. 50 degrees C. To this mixture was added 343.2 gm of cyanoacetic acid and 0.5 L of acetic anhydride and this homogeneous mixture was-stirred at 80-85 degrees C. for three hours. The mixture was cooled and concentrated under vacuum to remove acetic acid and residual acetic anhydride. The residue was triturated successively with 1.0 L portions of water, acetonitrile, toluene and ethyl acetate. The residue was then dried under vacuum to yield 1261 gm of a 2:1 mixture of 1′-cyanoacetyl-1-[2-(4-nitrophenyl)ethyl]-1′-propylurea (3) and its undesired isomer 1-cyanoacetyl-1-[2-(4-nitrophenyl)ethyl]-1′-propylurea. This material was dissolved in 2.2 L of hot ethyl acetate to which ca. 750 mL of hexanes were added to the cloud point and the mixture was allowed to cool to room temperature to induce crystallization. Filtration of the solid and air drying yielded 363 gm of 1′-cyanoacetyl-1-[2-(4-nitrophenyl)ethyl]-1′-propylurea (3). If needed, additional recrystallizations from ethyl acetate-hexanes could be carried out to provide pure 1′-cyanoacetyl-1-[2-(4-nitrophenyl)ethyl]-1′-propylurea (3).

### Step c: Conversion of 1′-Cyanoacetyl-1-L-(4-nitrophenyl)ethyl]-1′-propylurea (3) to 6-Amino-1-[2-(4-nitrophenyl)ethyl]-3-propyluracil (4)

A mixture of ca. 2N sodium hydroxide was produced by dissolving 336 gm of solid sodium hydroxide in 4.2 L of water. To this warm solution was added, in portions, 312 gm of 1′-cyanoacetyl-1-[2-(4-nitrophenyl)ethyl]-1′-propylurea (3) and the mixture was-stirred for 1 hour at 80 degrees C., then was cooled to room temperature with stirring to induce crystallization. The solids were isolated by filtration, washed with four 500 mL portions of water and vacuum dried at 65 degrees C. to yield 232 gm of crude 6-amino-1-[2-(4-nitrophenyl)ethyl]-3-propyluracil (4).

### Step d: Conversion of 6-Amino-1-[2-(4-nitrophenyl)ethyl]-3-propyluracil (4) to 6-Amino-5-nitroso-1-[2-(4-nitrophenyl)ethyl]-3-propyluracil (5)

To a solution of 232 gm of crude 6-amino-1-[2-(4-nitrophenyl)ethyl]-3-propyluracil (4), 4.0 L of water and ca. 2.0 L of ethanol at 80 degrees C. was added 55.3 gm of sodium nitrite in one portion, followed by the dropwise addition of 100 mL of glacial acetic acid. After stirring at 80 degrees C. for 20 minutes the mixture was allowed to cool to near room temperature, then was chilled in an ice bath to effect crystallization. The solids were isolated by filtration, washed with two 1.0 L portions of water and dried under vacuum to yield 244 gm of purple colored 6-amino-5-nitroso-1-[2-(4-nitrophenyl)ethyl]-3-propyluracil (5).

### Step e: Conversion of 6-Amino-5-nitroso-1-[2-(4-nitrophenyl)ethyl]-3-propyluracil (5) to 5,6-Diamino-1-[2-(4-nitrophenyl)ethyl]-3-propyluracil (6)

A mixture of 243 gm of 6-amino-5-nitroso-1-[2-(4-nitrophenyl)ethyl]-3-propyluracil (5), and 2.1 L of water was heated to reflux and 528 mL of a 50% aqueous solution of ammonium sulfide was added with stirring to control foaming. The dark solution was stirred at 90-100 degrees C. for 30 min. and allowed to cool with stirring for 1.5 hours. The mixture was then chilled in an ice bath to fully effect crystallization. The solids were isolated by filtration, washed with three 500 mL portions of water and dried under vacuum to yield 219 gm of a dark solid. This material was recrystallized from 1.0 L of acetonitrile to yield two crops totaling 169.5 gm of 5,6-diamino-1-[2-(4-nitrophenyl)ethyl]-3-propyluracil (6).

### EXAMPLE 2

### Synthesis of 8-Benzyl-3-[2-4-nitrophenyl)ethyl]-1-propylxanthine (9)

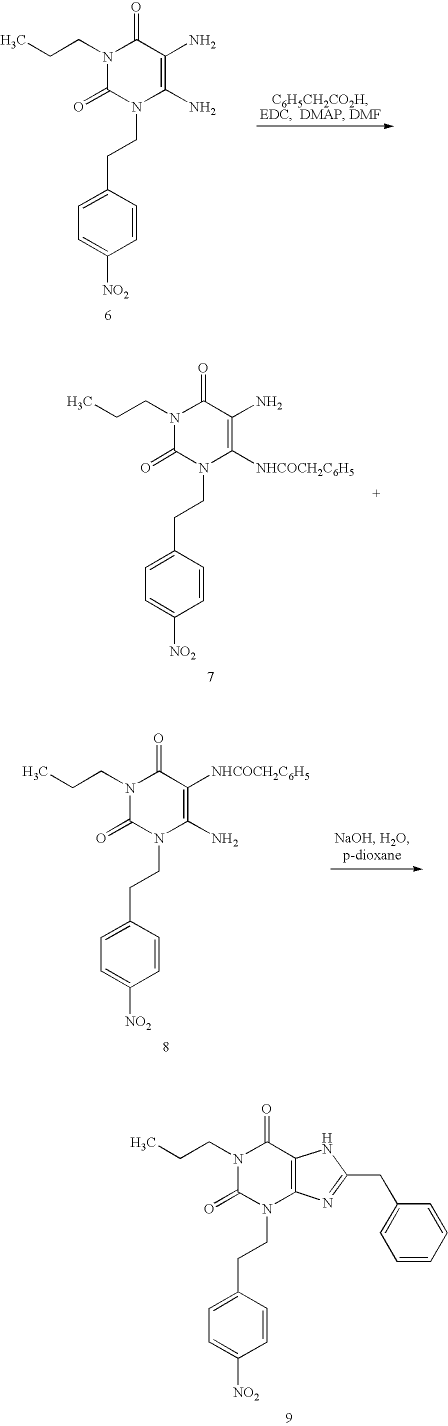

A solution of 44.9 gm of phenylacetic acid in 630 mL of dimethylformamide (DMF) was chilled in an ice water bath and 63.38 gm of 1-(3-dimethylaminopropyl)-3-ethylcarbodiimide hydrochloride (EDC) was added followed by 5.24 gm of 4-dimethylaminopyridine (DMAP). This mixture was stirred at ca. 4 degrees C. for 30 minutes and 100 gm of 5,6-diamino-1-[2-(4-nitrophenyl)ethyl]-3-propyluracil (6) was added in one portion. This mixture was stirred for 60 hr at room temperature. The dark homogeneous solution was poured into 700 mL of ice water with stirring to effect precipitation. The solids were isolated by filtration, washed with three 100 mL portions of water and dried under vacuum to yield 103 gm of a mixture of 5-amino-1-[2-(4-nitrophenyl)ethyl]-6-phenacetoamino-3-propyluracil (7) and 6-amino-1-[2-(4-nitrophenyl)ethyl]-5-phenacetoamino-3-propyluracil (8) intermediates. These solids were dissolved in 450 mL of p-dioxane, 600 mL of 2N aqueous sodium hydroxide was added and the mixture was heated at reflux for one hr. The solution was then chilled in an ice water bath and the pH adjusted to pH 4 with ca. 100 mL of concentrated hydrochloric acid to yield a precipitate. The solids were isolated by filtration, washed with three 100 mL portions of water and dried under vacuum to yield 82 gm of an orange solid. Recrystallization from hot ethyl acetate afforded 58.0 gm of 8-benzyl-3-[2-(4-nitrophenyl)ethyl]-1-propylxanthine (9).

### EXAMPLE 3

### Synthesis of 8-Benzyl-7-[2-ethyl(2-hydroxyethyl)amino]ethyl-3-[2-(4-nitrophenyl)ethyl]-1-propylxanthine

A mixture of 2.1 gm of 8-benzyl-3-[2-(4-nitrophenyl)ethyl]-1-propylxanthine (9), 1.02 gm of sodium carbonate, 3.82 ml of 1,2-dichloroethane and 0.59 ml of 2-(ethylamino)ethanol was heated in a steel pressure vessel under argon at 120 degrees C. for 3-5 hours. The mixture was then cooled and vented to the atmosphere. The semisolid reaction mixture was triturated several times with 5-10 ml portions of methanol followed by methylene chloride and the combined solutions were evaporated to dryness. The residue was purified by column chromatography on silica gel using a gradient of 1:1 ethyl acetate-hexanes, ethyl acetate and 5% methanol in ethyl acetate. The appropriate fractions were collected and evaporated to dryness to yield a light orange solid of 8-benzyl-7-[2-ethyl(2-hydroxyethyl)amino]ethyl-3-[2-(4-nitrophenyl)ethyl]-1-propylxanthine.

### EXAMPLE 4

### Synthesis of 3-[2-(4-Aminophenyl)ethyl]-8-benzyl-7-[2-ethyl(2-hydroxyethyl)amino]ethyl-1-propylxanthine Free Base and 3-[2-(4-Aminophenyl)ethyl]-8-benzyl-7-[2-ethyl(2-hydroxyethyl)-amino]ethyl-1-propylxanthine Dihydrochloride Salt

To a mixture of 9.4 gm of 8-benzyl-7-[2-ethyl(2-hydroxyethyl)amino]ethyl-3-[2-(4-nitrophenyl)ethyl]-1-propylxanthine in 400 ml of tetrahydrofuran under inert gas was added 1.2 gm of 10% palladium on carbon catalyst followed by the dropwise addition of 12 ml of hydrazine hydrate. The mixture was stirred for 2 hours at which time gas evolution subsided. An additional 600 mg of 10% palladium on carbon catalyst was added, followed by 5 ml of additional hydrazine hydrate. Additional catalyst and hydrazine hydrate were added as needed to complete the reaction. The reaction mixture was then filtered through Celite and evaporated to dryness to yield an orange oil. Purification by silica gel column chromatography afforded purified solid 3-[2-(4-aminophenyl)ethyl]-8-benzyl-7-[2-ethyl(2-hydroxyethyl)-amino]ethyl-1-propylxanthine free base which was dissolved in 75 ml of tetrahydrofuran. To this solution was added 15 ml of 4N hydrogen chloride in p-dioxane, which gave a white precipitate. This precipitate was stirred as a slurry, collected by filtration and vacuum dried to afford 3-[2-(4-aminophenyl)ethyl]-8-benzyl-7-[2-ethyl(2-hydroxyethyl)-amino]ethyl-1-propylxanthine dihydrochloride salt, m.p. 230-231 degrees C. (uncorrected).

### EXAMPLE 5

### Synthesis of 3-2-(4-Aminophenyl)ethyl]-8-benzyl-7-(2-ethylamino)ethyl-1-propylxanthine Free Base or Dihydrochloride Salt

By the method of Example 3, 8-benzyl-3-[2-(4-nitrophenyl)ethyl]-1-propylxanthine, is reacted with sodium carbonate, 1,2-dichloroethane and ethylamine to yield 8-benzyl-7-(2-ethylamino)ethyl-3-[2-(4-nitrophenyl)ethyl]-1-propylxanthine. By the method of Example 4, 8-benzyl-7-(2-ethylamino)ethyl-3-[2-(4-nitrophenyl)ethyl]-1-propylxanthine is reduced with hydrazine hydrate or hydrogen gas in the presence of a palladium catalyst to yield 3-[2-(4-aminophenyl)ethyl]-8-benzyl-7-(2-ethylamino)ethyl-1-propylxanthine free base. The corresponding dihydrochloride salt is then made on exposure to an excess of hydrogen chloride in solution.

### EXAMPLE 6

### Synthesis of 3-[2-(2-Aminophenyl)ethyl]-8-benzyl-7-[2-ethyl(2-hydroxyethyl)amino]ethyl-1-propylxanthine Free Base and 3-[2-(2-Aminophenyl)ethyl]-8-benzyl-7-[2-ethyl(2-hydroxyethyl)amino]ethyl-1-propylxanthine Dihydrochloride Salt

By the method of Example 1-, 2-nitrophenethylamine hydrochloride and n-propylisocyanate are converted into 5,6-diamino-1-2-(2-nitrophenyl)ethyl]-3-propyluracil. By the method of Example 2, phenylacetic acid is reacted with 5,6-diamino-1-[2-(2-nitrophenyl)ethyl]-3-propyluracil to yield 8-benzyl-3-[2-(2-nitrophenyl)ethyl]-1-propylxanthine. By the method of Example 3, this substance is alkylated with a mixture of 1,2-dichloroethane and 2-(ethylamino)ethanol to yield 8-benzyl-7-[2-ethyl(2-hydroxyethyl)amino]ethyl-3-[2-(2-nitrophenyl)ethyl]-1-propylxanthine. By the method of Example 4 this substance is reduced with hydrazine hydrate or hydrogen gas in the presence of a palladium catalyst to yield 3-[2-(2-aminophenyl)ethyl]-8-benzyl-7-[2-ethyl(2-hydroxyethyl)amino]ethyl-1-propylxanthine free base. The corresponding dihydrochloride salt is then made on exposure to an excess of hydrogen chloride in solution.

### EXAMPLE 7

### Synthesis of 3-[2-(3-Aminophenyl)ethyl]-8-benzyl-7-[2-ethyl(2-hydroxyethyl)-amino]ethyl-1-propylxanthine Free Base and 3-[2-(3-Aminophenyl)ethyl]-8-benzyl-7-[2-ethyl(2-hydroxyethyl)-amino]ethyl-1-propylxanthine Dihydrochloride Salt

By the method of Example 1, 3-nitrophenethylamine hydrochloride and n-propylisocyanate are converted into 5,6-diamino-1-[2-(3-nitrophenyl)ethyl]-3-propyluracil. By the method of Example 2, phenylacetic acid is reacted with 5,6-diamino-1-[2-(3-nitrophenyl)ethyl]-3-propyluracil to yield 8-benzyl-3-[2-(3-nitrophenyl)ethyl]-1-propylxanthine. By the method of Example 3, this substance is alkylated with a mixture of 1,2-dichloroethane and 2-(ethylamino)ethanol to yield 8-benzyl-7-[2-ethyl(2-hydroxyethyl)amino]ethyl-3-[2-(3-nitrophenyl)ethyl]-1-propylxanthine. By the method of Example 4 this substance is reduced with hydrazine hydrate or hydrogen gas in the presence of a palladium catalyst to yield 3-[2-(3-aminophenyl)ethyl]-8-benzyl-7-[2-ethyl(2-hydroxyethyl)amino]ethyl-1-propylxanthine free base. The corresponding dihydrochloride salt is then made on exposure to an excess of hydrogen chloride in solution.

### EXAMPLE 8

### Synthesis of 3-[2-(2-Aminophenyl)ethyl]-8-benzyl-7-(2-ethylamino)ethyl-1-propylxanthine Free Base or Dihydrochloride Salt

By the method of Example 3, 8-benzyl-3-[2-(2-nitrophenyl)ethyl]-1-propylxanthine, is reacted with sodium carbonate, 1,2-dichloroethane and ethylamine to yield 8-benzyl-7-(2-ethylamino)ethyl-3-[2-(2-nitrophenyl)ethyl]-1-propylxanthine. By the method of Example 4, 8-benzyl-7-(2-ethylamino)ethyl-3-[2-(2-nitrophenyl)ethyl]-1-propylxanthine is reduced with hydrazine hydrate or hydrogen gas in the presence of a palladium catalyst to yield 3-[2-(2-aminophenyl)ethyl]-8-benzyl-7-(2-ethylamino)ethyl-1-propylxanthine free base. The corresponding dihydrochloride salt is then made on exposure to an excess of hydrogen chloride in solution.

### EXAMPLE 9

### Synthesis of 3-[2-(3-Aminophenyl)ethyl]8-benzyl-7-(2-ethylamino)ethyl-1-propylxanthine Free Base or Dihydrochloride Salt

By the method of Example 3, 8-benzyl-3-[2-(3-nitrophenyl)ethyl]-1-propylxanthine, is reacted with sodium carbonate, 1,2-dichloroethane and diethylamine to yield 8-benzyl-7-(2-ethylamino)ethyl-3-[2-(3-nitrophenyl)ethyl-1-propylxanthine. By the method of Example 4, 8-benzyl-7-(2-ethylamino)ethyl-3-[2-(3-nitrophenyl)ethyl]-1-propylxanthine is reduced with hydrazine hydrate or hydrogen gas in the presence of a palladium catalyst to yield 3-[2-(3-aminophenyl)ethyl]-8-benzyl-7-(2-ethylamino)ethyl-1-propylxanthine free base. The corresponding dihydrochloride salt is then made on exposure to an excess of hydrogen chloride in solution.

### EXAMPLE 10

### Synthesis of Tritium Labelled 3-[2-(3-Aminophenyl)ethyl]-8-benzyl-7-[2-ethyl(2-hydroxyethyl)-amino]ethyl-1-propylxanthine Free Base and 3-[2-(3-Aminophenyl)ethyl]-8-benzyl-7-[1³H,2³H-[2-ethyl(2-hydroxyethyl)-amino]ethyl]-1-propylxanthine Dihydrochloride Salt

By the method of Example 1′, 3-nitrophenethylamine hydrochloride and n-propylisocyanate are converted into 5,6-diamino-1-[2-(3-nitrophenyl)ethyl]-3-propyluracil. By the method of Example 2, phenylacetic acid is reacted with 5,6-diamino-1-[2-(3-nitrophenyl)ethyl]-3-propyluracil to yield 8-benzyl-3-[2-(3-nitrophenyl)ethyl]-1-propylxanthine. By the method of Example 3, this substance is alkylated with a mixture of tritium-labeled 1,2-dichloroethane (³H-1,2-dichloroethane] and 2-(ethylamino)ethanol to yield tritium-labeled 8-benzyl-7-[1³H,2³H-[2-ethyl(2-hydroxyethyl)amino]ethyl]-3-[2-(3-nitrophenyl)ethyl]-1-propylxanthine. By the method of Example 4 this substance is reduced with hydrazine hydrate or hydrogen gas in the presence of a palladium catalyst to yield tritium-labeled 3-[2-(3-aminophenyl)ethyl]-8-benzyl-7-[1³H,2³H-[2-ethyl(2-hydroxyethyl)amino]ethyl]-1-propylxanthine free base. The corresponding tritium-labeled dihydrochloride salt is then made on exposure to an excess of hydrogen chloride in solution.

### EXAMPLE 11

### Synthesis of 3-[2-[4-(6-Aminohexanoyl)aminophenyl]ethyl]8-benzyl-7-[2-ethyl(2-hydroxyethyl)amino]ethyl-1-propylxanthine

By methods well known in the art, 3-[2-(4-aminophenylethyl]-8-benzyl-7-[2-ethyl(2-hydroxyethyl)amino]ethyl-1-propylxanthine is reacted with 6-aminohexanoic acid and a coupling agent such as 1-(3-dimethylaminopropyl)-3-ethylcarbodiimide hydrochloride (EDC) to yield 3-[2-[4-(6-aminohexanoyl)aminophenyl]ethyl]-8-benzyl-7-[2-ethyl(2-hydroxyethyl)amino]ethyl-1-propylxanthine.

### EXAMPLE 12

### Synthesis of 8-Benzyl-3-[2-[4-(3-carboxypropanoyl)aminophenyl]ethyl]-7-[2-ethyl(2-hydroxyethyl)amino]ethyl-1-propylxanthine

By methods well known in the art, 3-[2-(4-aminophenyl)ethyl]-8-benzyl-7-[2-ethyl(2-hydroxyethyl)amino]ethyl-1-propylxanthine is reacted with succinyl anhydride and a base such as triethylamine to yield 8-benzyl-3-[2-[4-(3-carboxypropanoyl)aminophenyl]ethyl]-7-[2-ethyl(2-hydroxyethyl)amino]ethyl-1-propylxanthine.

### EXAMPLE 13

### Synthesis of 3-[2-[4-(6-Aminohexyl-3-amidocarboxypropanoyl)-aminophenyl]ethyl]-8-benzyl-7-[2-ethyl(2-hydroxyethyl)amino]ethyl-1-propylxanthine

By methods well known in the art, 8-benzyl-3-[2-[4-(3-carboxypropanoyl)-aminophenyl]ethyl]-7-[2-ethyl(2-hydroxyethyl)amino]ethyl-1-propylxanthine is reacted with 1,6-diaminohexane and a coupling agent such as 1-(3-dimethylaminopropyl)-3-ethylcarbodiimide hydrochloride (EDC) to yield 3-(2-[4-(6-aminohexyl-3-amidocarboxypropanoyl)aminophenyl]ethyl]-8-benzyl-7-[2-ethyl(2-hydroxyethyl)amino]ethyl-1-propylxanthine.

### EXAMPLE 14

### Synthesis of the Cy3B-Coupled Amido Derivative of 3-[2-[4-(6-Aminohexyl-3-amidocarboxypropanoyl)aminophenyl]ethyl]-8-benzyl-7-[2-ethyl(2-hydroxyethyl)amino]ethyl-1-propylxanthine

By methods well known in the art, 3-[2-[4-(6-aminohexyl-3-amidocarboxypropanoyl)aminophenyl]ethyl]-8-benzyl-7-[2-ethyl(2-hydroxyethyl)amino]ethyl-1-propylxanthine is reacted with the commercially available 6,7,9,10-tetrahydro-2-carboxymethyl-14-sulfonato-16,16,18,18-tetramethyl-7aH,8aH-bisindolinium[3,2-a,3′2′-a]pyrano[3,2-c; 5,6-c′]dipyridin-5-ium, N-hydroxysuccinimidyl ester (sold as Cy3B by Amersham Biosciences, UK Limited, Little Chalfont, Buckinghamshire, England) and a base such as diisopropylethylamine to yield the Cy3B-coupled amido derivative of 3-[2-[4-(6-aminohexyl-3-amidocarboxypropanoyl)aminophenyl]ethyl]-8-benzyl-7-[2-ethyl(2-hydroxyethyl)amino]ethyl-1-propylxanthine.

### EXAMPLE 15

### Synthesis of 8-Benzyl-7-[2-ethyl(2-hydroxyethyl)amino]ethyl-3-[2-[4-(6-maleimidohexyl-3-amidocarboxypropanoyl)aminophenyl]ethyl]-1-propylxanthine

By methods well known in the art, 1,6-diaminohexane is reacted with maleic anhydride to produce 6-aminohexylmaleimide. By methods well, known in the art, 3-[2-[4-(3-carboxypropanoyl)aminophenyl]ethyl]-8-benzyl-7-[2-ethyl(2-hydroxyethyl)amino]ethyl-1-propylxanthine is, reacted with 6-aminohexylmaleimide and a coupling agent such as 1-(3-dimethylaminopropyl)-3-ethylcarbodiimide hydrochloride (EDC) to yield, after aqueous work-up, 8-benzyl-7-[2-ethyl(2-hydroxyethyl)amino]ethyl-3-[2-[4-(6-maleimidohexyl-3-amidocarboxypropanoyl)aminophenyl]ethyl]-1-propylxanthine.

### EXAMPLE 16

### Synthesis of 8-Benzyl-7-[2-ethyl(2-hydroxyethyl)amino]ethyl-3-[2-[4-(6-thiohexyl-3-amidocarboxypropanoyl)aminophenyl]ethyl]-1-propylxanthine

By methods well known in the art, 3-[2-[4-(3-carboxypropanoyl)aminophenyl]-ethyl]-8-benzyl-7-[2-ethyl(2-hydroxyethyl)amino]ethyl-1-propylxanthine is reacted with 6-aminohexanethiol and a coupling agent such as 1-(3-dimethylaminopropyl)-3-ethylcarbodiimide hydrochloride (EDC) to yield 8-benzyl-7-[2-ethyl(2-hydroxyethyl)amino]ethyl-3-[2-[4-(6-thiohexyl-3-amidocarboxypropanoyl)aminophenyl]ethyl]-1-propylxanthine.

### EXAMPLE 17

### Synthesis of the Cy3B-Coupled Thio Derivative of 8-Benzyl-7-[2-ethyl(2-hydroxyethyl)-amino]ethyl-3-[2-[4-(6-thiohexyl-3-amidocarboxy-propanoyl)aminophenyl]ethyl]-1-propylxanthine

By methods well known in the art, 8-benzyl-7-[2-ethyl(2-hydroxyethyl)-amino]ethyl-3-[2-[4-(6-thiohexyl-3-amidocarboxypropanoyl)aminophenyl]ethyl]-1-propylxanthine is reacted with the commercially available 6,7,9,10-tetrahydro-2-carboxymethyl-14-sulfonato-16,16,18,18-tetramethyl-7aH,8aH-bisindolinium[3,2-a,3′2′-a]pyrano[3,2-c; 5,6-c′]dipyridin-5-ium, N-hydroxysuccinimidyl ester (sold as Cy3B by Amersham Biosciences UK Limited, Little Chalfont, Buckinghamshire, England) and a base such as diisopropylethylamine to yield the Cy3B-coupled thio derivative of 8-benzyl-7-[2-ethyl(2-hydroxyethyl)amino]ethyl-3-[2-[4-(6-thiohexyl-3-amidocarboxypropanoyl)aminophenyl]ethyl]-1-propylxanthine.

### EXAMPLE 18

In like manner to the previous Examples 1-9, the following compounds are prepared:

3-[2-(2-Aminophenyl)ethyl]-8-benzyl-7-(2-methylamino)ethyl-1-propylxanthine,

3-[2-(3-Aminophenyl)ethyl]-8-benzyl-7-(2-methylamino)ethyl-1-propylxanthine,

3-[2-(4-Aminophenyl)ethyl]-8-benzyl-7-(2-methylamino)ethyl-1-propylxanthine,

3-[2-(2-Aminophenyl)ethyl]-8-benzyl-7-(2-ethylamino)ethyl-1-propylxanthine,

3-[2-(3-Aminophenyl)ethyl]-8-benzyl-7-(2-ethylamino)ethyl-1-propylxanthine,

3-[2-(4-Aminophenyl)ethyl]-8-benzyl-7-(2-ethylamino)ethyl-1-propylxanthine,

3-[2-(2-Aminophenyl)ethyl]-7-(2-ethylamino)ethyl-8-[4-(2-hydroxyethyl)]benzyl-1-propylxanthine,

3-[2-(3-Aminophenyl)ethyl]-7-(2-ethylamino)ethyl-8-[4-(2-hydroxyethyl)]benzyl-1-propylxanthine,

3-[2-(4-Aminophenyl)ethyl]-7-(2-ethylamino)ethyl-8-[4-(2-hydroxyethyl)]benzyl-1-propylxanthine,

8-(4-Aminobenzyl)-3-[2-(2-aminophenyl)ethyl]-7-[2-ethylamino]ethyl-1-propylxanthine,

8-(4-Aminobenzyl)-3-[2-(3-aminophenyl)ethyl]-7-[2-ethylamino]ethyl-1-propylxanthine,

3-[2-(2-Aminophenyl)ethyl]-8-benzyl-7-(2-ethylamino)ethyl-1-propylxanthine, and

3-[2-(3-Aminophenyl)ethyl]-8-benzyl-7-(2-ethylamino)ethyl-1-propylxanthine.

### EXAMPLE 19

In like manner to the previous Examples 1-9, the following compounds are prepared:

3-[2-(2-Aminophenyl)ethyl]-8-benzyl-7-[2-methyl(2-hydroxyethyl)amino]ethyl-1-propylxanthine,

3-[2-(3-Aminophenyl)ethyl]-8-benzyl-7-[2-methyl(2-hydroxyethyl)amino]ethyl-1-propylxanthine,

3-[2-(2-Aminophenyl)ethyl]-8-benzyl-7-[2-ethyl(2-hydroxyethyl)amino]ethyl-1-propylxanthine,

3-[2-(3-Aminophenyl)ethyl]-8-benzyl-7-[2-ethyl(2-hydroxyethyl)amino]ethyl-1-propylxanthine,

8-(4-Aminobenzyl)-3-[2-(2-aminophenyl)ethyl]-7-[2-ethyl(2-hydroxyethyl)amino]ethyl-1-propylxanthine, and

8-(4-Aminobenzyl)-3-[2-(3-aminophenyl)ethyl]-7-[2-ethyl(2-hydroxyethyl)amino]ethyl-1-propylxanthine.

### EXAMPLE 20

In like manner to the previous Examples 1-9, the following compounds are prepared:

3-[4-(2-Aminophenyl)butyl]-8-benzyl-7-[2-ethyl(2-hydroxyethyl)amino]ethyl-1-propylxanthine,

3-[6-(2-Aminophenyl)hexyl]-8-benzyl-7-[2-ethyl(2-hydroxyethyl)-amino]ethyl-1-propylxanthine,

3-[4-(2-Aminophenyl)butyl]-8-benzyl-7-(2-ethylamino)ethyl-1-propylxanthine,

3-[6-(2-Aminophenyl)hexyl]-8-benzyl-7-(2-ethylamino)ethyl-1-propylxanthine,

3-[4-(3-Aminophenyl)butyl]-8-benzyl-7-[2-ethyl(2-hydroxyethyl)amino]ethyl-1-propylxanthine,

3-[6-(3-Aminophenyl)hexyl]-8-benzyl-7-[2-ethyl(2-hydroxyethyl)-amino]ethyl-1-propylxanthine,

3-[4-(3-Aminophenyl)butyl]-8-benzyl-7-(2-ethylamino)ethyl-1-propylxanthine,

3-[6-(3-Aminophenyl)hexyl]-8-benzyl-7-(2-ethylamino)ethyl-1-propylxanthine,

3-[4-(4-Aminophenyl)butyl]-8-benzyl-7-[2-ethyl(2-hydroxyethyl)-amino]ethyl-1-propylxanthine,

3-[6-(4-Aminophenyl)hexyl]-8-benzyl-7-[2-ethyl(2-hydroxyethyl)-amino]ethyl-1-propylxanthine,

3-[4-(4-Aminophenyl)butyl]-8-benzyl-7-(2-ethylamino)ethyl-1-propylxanthine, and

3-[6-(4-Aminophenyl)hexyl]-8-benzyl-7-(2-ethylamino)ethyl-1-propylxanthine.

### EXAMPLE 21

In like manner to the previous Example 12, the following compounds are prepared:

8-Benzyl-3-[2-[2-(3-carboxypropanoyl)aminophenyl]ethyl]-7-[2-ethyl(2-hydroxyethyl)amino]ethyl-1-propylxanthine,

8-Benzyl-3-[2-[3-(3-carboxypropanoyl)aminophenyl]ethyl]-7-[2-ethyl(2-hydroxyethyl)amino]ethyl-1-propylxanthine,

8-Benzyl-3-[2-[4-(3-carboxypropanoyl)aminophenyl]ethyl]-7-[2-ethyl(2-hydroxyethyl)amino]ethyl-1-propylxanthine,

8-Benzyl-3-[2-[2-(3-carboxypropanoyl)aminophenyl]ethyl]-7-(2-ethylamino)ethyl-1-propylxanthine,

8-Benzyl-3-[2-[2-(3-carboxypropanoyl)aminophenyl]ethyl]-7-(2-ethylamino)ethyl-1-propylxanthine, and

8-Benzyl-3-[2-[4-(3-carboxypropanoyl)aminophenyl]ethyl]-7-(2-ethylamino)ethyl-1-propylxanthine.

### EXAMPLE 22

In like manner to the previous Example 11, the following compounds are prepared:

3-[2-[2-(6-Aminohexanoyl)aminophenyl]ethyl]-8-benzyl-7-[2-ethyl(2-hydroxyethyl)amino]ethyl-1-propylxanthine,

3-[2-[3-(6-Aminohexanoyl)aminophenyl]ethyl]-8-benzyl-7-[2-ethyl(2-hydroxyethyl)amino]ethyl-1-propylxanthine,

3-[2-[4-(6-Aminohexanoyl)aminophenyl]ethyl]-8-benzyl-7-[2-ethyl(2-hydroxyethyl)amino]ethyl-1-propylxanthine,

3-[2-[2-(6-Aminohexanoyl)aminophenyl]ethyl]-8-benzyl-7-(2-ethylamino])ethyl-1-propylxanthine,

3-[2-[3-(6-Aminohexanoyl)aminophenyl]ethyl]-8-benzyl-7-(2-ethylamino)ethyl-1-propylxanthine, and

3-[2-[4-(6-Aminohexanoyl)aminophenyl]ethyl]-8-benzyl-7-(2-ethylamino)ethyl-1-propylxanthine.

### EXAMPLE 23

In like manner to the previous Example 13, the following compounds are prepared:

3-[2-[2-(6-Aminohexyl-3-amidocarboxypropanoyl)aminophenyl]ethyl]-8-benzyl-7-[2-ethyl(2-hydroxyethyl)amino]ethyl-1-propylxanthine,

3-[2-[3-(6-Aminohexyl-3-amidocarboxypropanoyl)aminophenyl]ethyl]-8-benzyl-7-[2-ethyl(2-hydroxyethyl)amino]ethyl-1-propylxanthine,

3-[2-[4-(6-Aminohexyl-3-amidocarboxypropanoyl)aminophenyl]ethyl]-8-benzyl-7-[2-ethyl(2-hydroxyethyl)amino]ethyl-1-propylxanthine,

3-[2-[2-(6-Aminohexyl-3-amidocarboxypropanoyl)aminophenyl]ethyl]-8-benzyl-7-(2-ethylamino)ethyl-1-propylxanthine,

3-[2-[3-(6-Aminohexyl-3-amidocarboxypropanoyl)aminophenyl]ethyl]-8-benzyl-7-(2-ethylamino)ethyl-1-propylxanthine, and

3-[2-[4-(6-Aminohexyl-3-amidocarboxypropanoyl)aminophenyl]ethyl]-8-benzyl-7-(2-ethylamino)ethyl-1-propylxanthine.

### EXAMPLE 24

In like manner to the previous Example 13, the following compounds are prepared:

3-[2-[2-(5-Aminopentyl-3-amidocarboxypropanoyl)aminophenyl]ethyl]-8-benzyl-7-[2-ethyl(2-hydroxyethyl)amino]ethyl-1-propylxanthine,

3-[2-[3-(5-Aminopentyl-3-amidocarboxypropanoyl)aminophenyl]ethyl]-8-benzyl-7-[2ethyl(2-hydroxyethyl)amino]ethyl-1-propylxanthine,

3-[2-[4-(5-Aminopentyl-3-amidocarboxypropanoyl)aminophenyl]ethyl]-8-benzyl-7-[2-ethyl(2-hydroxyethyl)amino]ethyl-1-propylxanthine,

3-[2-[2-(5-Aminopentyl-3-amidocarboxypropanoyl)aminophenyl]ethyl]-8-benzyl-7-(2-ethylamino)ethyl-1-propylxanthine,

3-[2-[3-(5-Aminopentyl-3-amidocarboxypropanoyl)aminophenyl]ethyl]-8-benzyl-7-(2-ethylamino)ethyl-1-propylxanthine, and

3-[2-[4-(5-Aminopentyl-3-amidocarboxypropanoyl)aminophenyl]ethyl]-8-benzyl-7-(2-ethylamino)ethyl-1-propylxanthine.

### EXAMPLE 25

In like manner to the previous Example 15, the following compounds are prepared:

8-Benzyl-7-[2-ethyl(2-hydroxyethyl)amino]ethyl-3-[2-[2-(6-N-maleimidohexyl-3-amidocarboxypropanoyl)aminophenyl]ethyl]-1-propylxanthine,

8-Benzyl-7-[2-ethyl(2-hydroxyethyl)amino]ethyl-3-[2-[3-(6-N-maleimidohexyl-3-amidocarboxypropanoyl)aminophenyl]ethyl]-1-propylxanthine,

8-Benzyl-7-[2-ethyl(2-hydroxyethyl)amino]ethyl-3-[2-[4-(6-N-maleimidohexyl-3-amidocarboxypropanoyl)aminophenyl]ethyl]-1-propylxanthine,

8-Benzyl-7-(2-ethylamino)ethyl-3-[2-[2-(6-N-maleimidohexyl-3-amidocarboxypropanoyl)aminophenyl]ethyl]-1-propylxanthine,

8-Benzyl-7-(2-ethylamino)ethyl-3-[2-[3-(6-N-maleimidohexyl-3-amidocarboxypropanoyl)aminophenyl]ethyl]-1-propylxanthine, and

8-Benzyl-7-(2-ethylamino)ethyl-3-[2-[4-(6-N-maleimidohexyl-3-amidocarboxypropanoyl)aminophenyl]ethyl]-1-propylxanthine.

### EXAMPLE 26

In like manner to the previous Example 16, the following compounds are prepared:

8-Benzyl-7-[2-ethyl(2-hydroxyethyl)amino]ethyl-1-propyl-3-[2-[2-(6-thiohexyl-3-amidocarboxypropanoyl)aminophenyl]ethyl]xanthine,

8-Benzyl-7-[2-ethyl(2-hydroxyethyl)amino]ethyl-1-propyl-3-[2-[3-(6-thiohexyl-3-amidocarboxypropanoyl)aminophenyl]ethyl]xanthine,

8-Benzyl-7-[2-ethyl(2-hydroxyethyl)amino]ethyl-1-propyl-3-[2-[4-(6-thiohexyl-3-amidocarboxypropanoyl)aminophenyl]ethyl]xanthine,

8-Benzyl-7-(2-ethylamino)ethyl-1-propyl-3-[2-[2-6-thiohexyl-3-amidocarboxypropanoyl)aminophenyl]ethyl]xanthine,

8-Benzyl-7-(2-ethylamino)ethyl-1-propyl-3-[2-[3-(6-thiohexyl-3-amidocarboxypropanoyl)aminophenyl]ethyl]xanthine, and

8-Benzyl-7-(2-ethylamino)ethyl-1-propyl-3-[2-[4-(6-thiohexyl-3-amidocarboxypropanoyl)aminophenyl]ethyl]xanthine.

### EXAMPLE 27

### Pharmaceutical Formulations

(A) Tablet

| | Amount per Tablet | |
|---|---|---|
| Active Ingredient: Compound of Formula (I) | 150 | mg |
| Starch | 50 | mg |
| Microcrystalline cellulose | 45 | mg |
| Polyvinylpryrrolidone (as 10% solution in water) | 5 | mg |
| Sodium carboxymethyl starch | 5 | mg |
| Magnesium stearate | 1 | mg |
| Talc | 1 | mg |

The active ingredient, starch and cellulose are passed through a No. 45 mesh U.S. sieve and mixed thoroughly. The aqueous solution containing polyvinylpyrrolidone is mixed with the resultant powder, and the mixture then is passed through a No. 14 mesh U.S. sieve. The granules so produced are dried at 50° C. and passed through a No. 18 mesh U.S. sieve. The sodium carboxymethyl starch, magnesium stearate and talc, previously passed through a No. 60 mesh U.S. sieve, are then added to the granules which, after mixing, are compressed in a tablet machine to yield tablets.

(B) Capsule

| | Amount per Capsule | |
|---|---|---|
| Active Ingredient: Compound of Formula (I) | 150 | mg |
| Starch | 24 | mg |
| Microcrystalline cellulose | 24 | mg |
| Magnesium stearate | 2 | mg |

The active ingredient, cellulose, starch and magnesium stearate are blended, passed through a No. 45 mesh U.S. Sieve, and filed into hard gelatin capsules.

(C) Intravenous Fluid

| | Amount per bag |
|---|---|
| Active Ingredient: Compound of Formula (I) | 100 mg |
| Sterile Isotonic saline for injection | 250 ml |

In a sterile environment, the active ingredient is dissolved in the isotonic saline and the resulting solution is passed through a 2 micron filter then filed into sterile intravenous fluid bags that are immediately sealed.

In the specification and examples, there have been disclosed typical embodiments of the invention and, although specific terms are employed, they are used in a generic and descriptive sense only and not for purposes of limitation of the scope of the invention being set forth in the following claims.

1. A compound of formula (I): 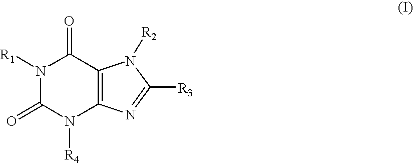
wherein
R₁ is a branched or straight chain C₁-C₈ alkyl;
R₂ is of the formula (II), 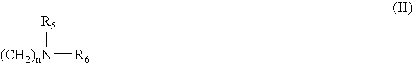
wherein n is an integer ranging from 1 to 8; R₅ is H or (CH₂)ₚCH₃, and R₆ is H or (CH₂)ₘOH,
wherein p is an integer ranging from 1 to 7 and m is an integer ranging from 1 to 8;
R₃ is of the formula (III),
(CH₂)_{q}C₆H₄—R₇ (III)
wherein q is an integer ranging from 1 to 8; and R₇ is selected from the group consisting of H, OH, NH₂, (CH₂)ₜOH, and R₉COOH;
wherein R₉ is a straight or branched chain alkylene or alkenylene group having 1 to 8 carbon atoms, and t is an integer ranging from 1 to 8;
R₄ is of the formula (IV), 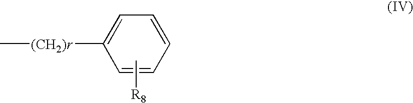
wherein r is an integer ranging from 1 to 8 and R₈ is ortho or meta and is selected from the group consisting of H, OH, (CH₂)_{f}NH₂, (CH₂)ₛOH, and R₁₀COOH
wherein f is 0 or f and s are independently integers ranging from 1 to 8; and,
R₁₀ is a C₁-C₈ straight or branched chain alkylene or alkenylene; and; salts, solvates, and hydrates thereof.

2. The compound according to claim 1, wherein R₁ is C₃ alkyl; R₂ is —(CH₂)₂N(CH₂CH₃)(CH₂)₂OH; R₈ is NH₂; R₇ is H; R₈ is NH₂; f is 0; n is 2; q is 1; and r is 2.

3. The compound according to claim 1, wherein R₁ is C₃ alkyl; R₅ is CH₃(CH₂)ₚ wherein p is 1; R₆ is H; R₇ is NH₂; f is 0; R₈ is NH₂; n is 2; q is 1; and r is 2.

4. The compound according to claim 1, wherein R₁ is C₃ alkyl; R₅ is CH₃(CH₂)ₚ wherein p is 1; R₆ is H; R₇ is H; R₈ is NH₂; f is 0; n is 2; q is 1; and r is 2.

5. The compound according to claim 1, wherein R₁ is C₃ alkyl; R₅ is CH₃(CH₂)ₚ wherein p is 1; R₆ is H; R₇ is H; R₈ is (CH₂)ₛOH wherein s is 2 and R₁₀COOH, wherein R₁₀ is CH═CH; n is 2; q is 1; and r is 2.

6. The compound according to claim 1, wherein R₁ is C₃ alkyl; R₅ is CH₃(CH₂)ₚ wherein p is 1; R₆ is H; R₇ is (CH₂)ₜOH wherein t is 2 and R₉COOH, wherein R₉ is CH═CH; R₈ is NH₂; f is 0; n is 2; q is 1; and r is 2.

7. A diagnostic assay-type probe comprising a compound according to claim 1: 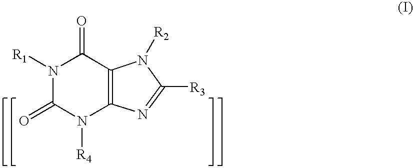 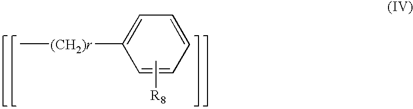
wherein the compound is labeled by a radioactive or non-radioactive material thereon or optionally connected to the compound of formula (I) by a spacer component present thereon, wherein the spacer component has functionality which bonds to the amine, hydroxyl, or carboxyl functionality present on the R₇ or R₈ substituent of the compound.

8. The assay-type probe according to claim 7, wherein said non-radioactive material is a fluorescent dye.

9. The assay-type probe according to claim 7, wherein said non-radioactive material is biotin.

10. The assay-type probe according to claim 7, wherein said non-radioactive material is a luminescent dye.

11. The assay-type probe according to claim 7, wherein said non-radioactive material is obelin.

12. The assay-type probe according to claim 7, wherein R₁ is C₃ alkyl, R₅ is CH₃(CH₂)ₚ; p is 1; R₇ is H; R₈ is NH₂; f is 0; n is 2; q is 1; r is 2; and R₆ is (CH₂)ₘOH; m is 2; and the non-radioactive material is biotin bonded to the hydroxyl group present on R₆.

13. The assay-type probe according to claim 7, wherein R₁ is C₃ alkyl; R₅ is CH₃(CH₂)ₚ wherein p is 1; R₆ is H; R₇ is NH₂; f is 0; n is 2; q is 1; r is 2; R₈ is NH₂; and the non-radioactive material is biotin bonded to the amino group present on R₈.

14. The assay-type probe according to claim 7, wherein R₁ is C₃ alkyl; R₅ is CH₃(CH₂)ₚ wherein p is 1; R₆ is H; R₇ is H; n is 2; q is 1; r is 2; and R₈ is R₁₀COOH, wherein R₁₀ is an alkylene or alkenylene group having 1 to 8 carbon atoms; and the non-radioactive material is biotin bonded to the carboxyl group present on R₈.

15. The assay-type probe according to claim 7, wherein the compound is labeled by the radioactive material connected by a spacer component, and the spacer component has functionality which bonds to the amine, hydroxyl, or carboxyl functionality present on the R₇ or R₈ substituent of the compound.

16. The assay-type probe according to claim 7, wherein the compound is labeled by the radioactive material and the radioactive material is a radioactive isotope selected from the group consisting of ¹⁸F, tritium, ¹¹C, ¹³C, and ¹⁵N; a complex of a metal atom or complex of a metal ion, a chelating agent, or ¹²⁵I.

17. An imaging agent for adenosine receptors comprising a compound according to claim 1; 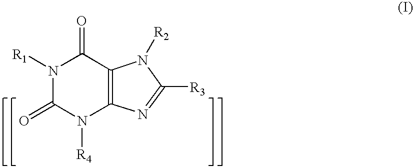 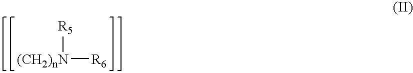
wherein at least one of its atoms or one or more atoms bonded thereto are radioactive, spin labeled, or both radioactive and spin labeled.

18. The imaging agent of according to claim 17 wherein the marker atom is a nuclear spin labeled.

19. The imaging agent of according to claim 18 wherein the marker atom is a ¹⁹F.

20. The imaging agent of according to claim 17 wherein the marker atom is a radioactive isotope.

21. The imaging agent of according to claim 17 wherein the radioactive isotope is ¹⁸F, ¹¹C, ¹⁵N, ¹²⁵I, or ³H

22. A method of treating A₁ adenosine receptor related disorders in a mammal in need of treatment thereof, comprising administering an effective amount of a compound according to claim 1,
or a pharmaceutically acceptable salt, solvate, or hydrate thereof, or a combination of compounds according to claim 1, optionally in combination with one or more other therapeutic agents, to the mammal in need thereof.

23. The method according to claim 22 wherein for the compound of formula (I) R₁ is C₃ alkyl; R₅ is CH₃(CH₂)ₚ wherein p is 1; R₆ is (CH₂)ₘOH wherein m is 2; R₇ is H; R₈ is NH₂; f is 0; n is 2; m is 2; q is 1; and r is 2.

24. The method according to claim 22 wherein for the compound of formula (I) R₁ is C₃ alkyl; R₅ is CH₃(CH₂)ₚ wherein p is 1; R₆ is H; R₇ is NH₂; R₈ is NH₂; f is 0; n is 2; q is 1; and r is 2.

25. The method according to claim 22 wherein for the compound of formula (I) R₁ is C₃ alkyl; R₅ is CH₃(CH₂)ₚ wherein p is 1; R₆ is H; R₇ is H; R₈ is NH₂; f is 0; n is 2; q is 1; and r is 2.

26. The method according to claim 22 wherein for the compound of formula (I) R₁ is C₃ alkyl; R₅ is CH₃(CH₂)ₚ wherein p is 1; R₆ is H; R₇ is H; R₈ is (CH₂)ₛOH wherein s is 2 and R₁₀COOH, wherein R₁₀ is CH═CH; n is 2; q is 1; and r is 2.

27. The method according to claim 22 wherein for the compound of formula (I) R₁ is C₃ alkyl; R₅ is CH₃(CH₂)ₚ wherein p is 1; R₆ is H; R₇ is (CH₂)ₜOH wherein t is 2 and R₉COOH, wherein R₉ is CH═CH; R₈ is NH₂; f is 0; n is 2; q is 1; and r is 2.

28. The method according to claim 22 wherein the A₁ adenosine receptor related disorder is congestive heart failure, hypertension, ischemia-reperfusion organ injury, endotoxin-related tissue injury, renal failure, Alzheimer's disease, depression, obesity, asthma, diabetes, cystic fibrosis, allergic conditions, autoimmune disorders, inflammatory disorders, chronic obstructive pulmonary disorders, chronic cough, coronary artery disease, biliary colic, postoperative ileus, fibrosis, sclerosis, Adult Respiratory Distress Syndrome (ARDS), acquired immunodeficiency syndrome (AIDS), Acute Lung Injury (ALI), acquired immunodeficiency syndrome (AIDS), Severe Acute Respiratory Syndrome (SARS), septicemia, substance abuse, drug dependence, or Parkinson's disease and the mammal is a human.

29. A pharmaceutical composition comprising a compound according to claim 1,
or a pharmaceutically salt, solvate, or hydrate thereof and a pharmaceutically acceptable carrier.

30. A prodrug comprising a compound according to claim 1.

31. A method of treating an A₁ adenosine receptor related disorder in a patient in need of treatment thereof, comprising administering an effective amount of a prodrug according to claim 30, or a pharmaceutically acceptable salt, solvate, or hydrate thereof, optionally in combination with one or more other therapeutic agents, to the patient in need thereof.

32. A method of preparing a compound of claim 1, the method comprising:
condensing R₄—NH₂ with R₁NCO to yield a substituted urea of formula (V) 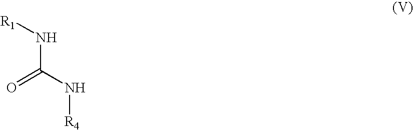
condensing a substituted urea of formula (V) with cyanoacetic acid to yield a compound of formula (VI), 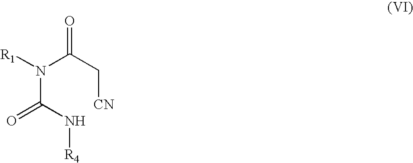
converting a compound of formula (VI) by treatment with strong base to yield a compound of formula (VII), 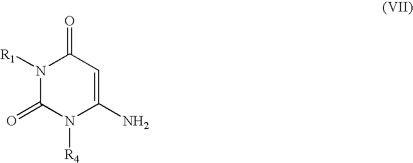
reacting a compound of formula (VII) with NaNO₂ under acidic conditions to yield a compound of formula (VIII) 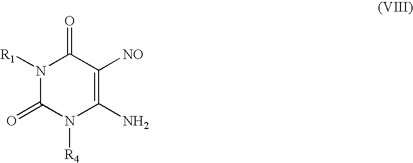
reducing a compound of formula (VIII) to produce a compound of formula (IX) 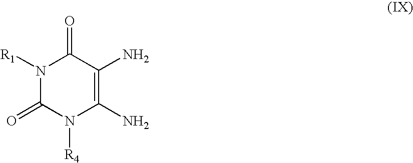
condensing a compound of formula (IX) with R₃—CO₂H a compound of either formula (Xa) or (Xb), 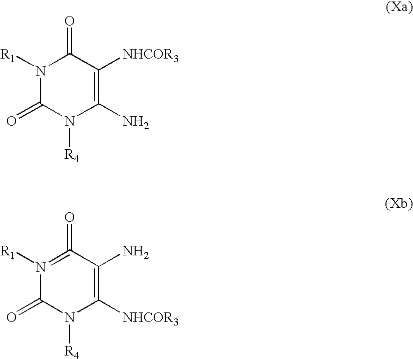
cyclizing a compound of formula (Xa) or (Xb) in the presence of a strong base to form a compound of formula (XI), and 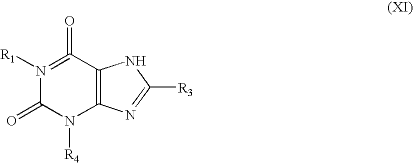
reacting a compound of formula (XI) with L-R2, wherein L is a leaving group, to yield a compound of formula (I).

33. The compound according to claim 1, wherein the compound is:
3-[2-(2-Aminophenyl)ethyl]-8-benzyl-7-(2-methylamino)ethyl-1-propylxanthine,
3-[2-(3-Aminophenyl)ethyl]-8-benzyl-7-(2-methylamino)ethyl-1-propylxanthine,
3-[2-(2-Aminophenyl)ethyl]-8-benzyl-7-(2-ethylamino)ethyl-1-propylxanthine,
3-[2-(3-Aminophenyl)ethyl]-8-benzyl-7-(2-ethylamino)ethyl-1-propylxanthine,
3-[2-(2-Aminophenyl)ethyl]-7-(2-ethylamino)ethyl-8-[4-(2-hydroxyethyl)]benzyl-1-propylxanthine,
3-[2-(3-Aminophenyl)ethyl]-7-(2-ethylamino)ethyl-8-[4-(2-hydroxyethyl)]benzyl-1-propylxanthine;
8-(4-Aminobenzyl)-3-[2-(2-aminophenyl)ethyl]-7-[2-ethylamino]ethyl-1-propylxanthine
8-(4-Aminobenzyl)-3-[2-(3-aminophenyl)ethyl]-7-[2-ethylamino]ethyl-1-propylxanthine,
3-[2-(2-Aminophenyl)ethyl]-8-benzyl-7-(2-ethylamino)ethyl-1-propylxanthine,
3-[2-(3-Aminophenyl)ethyl]-8-benzyl-7-(2-ethylamino)ethyl-1-propylxanthine,
3-[2-(2-Aminophenyl)ethyl]-8-benzyl-7-[2-methyl(2-hydroxyethyl)amino]ethyl-1-propylxanthine,
3-[2-(3-Aminophenyl)ethyl]-8-benzyl-7-[2-methyl(2-hydroxyethyl)amino]ethyl-1-propylxanthine,
3-[2-(2-Aminophenyl)ethyl]-8-benzyl-7-[2-ethyl(2-hydroxyethyl)amino]ethyl-1-propylxanthine,
3-[2-(3-Aminophenyl)ethyl]-8-benzyl-7-[2-ethyl(2-hydroxyethyl)amino]ethyl-1-propylxanthine,
8-(4-Aminobenzyl)-3-[2-(2-aminophenyl)ethyl]-7-[2-ethyl(2-hydroxyethyl)amino]ethyl-1-propylxanthine,
8-(4-Aminobenzyl)-3-[2-(3-aminophenyl)ethyl]-7-[2-ethyl(2-hydroxyethyl)amino]ethyl-1-propylxanthine,
3-[4-(2-Aminophenyl)butyl]-8-benzyl-7-[2-ethyl(2-hydroxyethyl)amino]ethyl-1-propylxanthine,
3-[6-(2-Aminophenyl)hexyl]-8-benzyl-7-[2-ethyl(2-hydroxyethyl)-amino]ethyl-1-propylxanthine,
3-[4-(2-Aminophenyl)butyl]-8-benzyl-7-(2-ethylamino)ethyl-1-propylxanthine,
3-[6-(2-Aminophenyl)hexyl]-8-benzyl-7-(2-ethylamino)ethyl-1-propylxanthine,
3-[4-(3-Aminophenyl)butyl]-8-benzyl-7-[2-ethyl(2-hydroxyethyl)amino]ethyl-1-propylxanthine,
3-[6-(3-Aminophenyl)hexyl]-8-benzyl-7-[2-ethyl(2-hydroxyethyl)-amino]ethyl-1-propylxanthine,
3-[4-(3-Aminophenyl)butyl]-8-benzyl-7-(2-ethylamino)ethyl-1-propylxanthine or
3-[6-(3-Aminophenyl)hexyl]-8-benzyl-7-(2-ethylamino)ethyl-1-propylxanthine.
